(19) United States
(12) Patent Application Publication
(10) Pub. No.: US 2024/0325723 A1
(43) Pub. Date: Oct. 3, 2024

(54) ECMO AND LV UNLOADING SYSTEM

(71) Applicant: ABIOMED, Inc., Danvers, MA (US)

(72) Inventor: Christopher Korkuch, Danvers, MA (US)

(73) Assignee: ABIOMED, Inc., Danvers, MA (US)

(21) Appl. No.: 18/435,065

(22) Filed: Feb. 7, 2024

Related U.S. Application Data
(60) Provisional application No. 63/444,408, filed on Feb. 9, 2023.

(51) Int. Cl. A61M 60/865; A61M 1/36; A61M 25/00; A61M 39/02; A61M 39/06; A61M 60/13

(52) U.S. Cl. CPC A61M 60/865; A61M 1/3659; A61M 1/3666; A61M 25/0013; A61M 39/06; A61M 60/13; A61M 2039/0258; A61M 2039/0273

(57) ABSTRACT

An unloading system may be provided for use with ECMO and mechanical circulatory support (MCS) devices. The system may include a first cannula with a distal cannula body coupled to a proximal hub, the distal body having an inner diameter, where the first cannula is configured to slidably receive the MCS device. The system may include a second cannula having an outer diameter smaller than the outer diameter of the first cannula. The system may include a connector for splitting a flow of blood from the ECMO device to the first and second cannula. The system may be configured so a first total pressure drop of the first cannula with the MCS device in place in the first cannula with the second cannula connected to the connector is equal to a second total pressure drop of the first cannula without the MCS device in place in the first cannula.

### CROSS-REFERENCE TO RELATED APPLICATIONS

The present application claims priority to U.S. 63/444,408, filed Feb. 9, 2023, the contents of which are incorporated by reference herein in its entirety.

### TECHNICAL FIELD

The present disclosure is drawn to techniques for providing patient support, such as providing both extracorporeal membrane oxygenation (ECMO) and mechanical circulatory support (MCS) to a patient through a single access site.

### BACKGROUND

This section is intended to introduce the reader to various aspects of art, which may be related to various aspects of the present invention that are described and/or claimed below. This discussion is believed to be helpful in providing the reader with background information to facilitate a better understanding of the various aspects of the present invention. Accordingly, it should be understood that these statements are to be read in this light, and not as admissions of prior art.

Delivery of combined ECMO (such as veno-arterial (VA) ECMO) and mechanical circulatory support, e.g., using a catheter-based blood pump such as an IMPELLA® heart pumps from Abiomed, Inc., currently requires two arterial access sites—one for the ECMO arterial return cannula and one for the MCS. However, both these sites are “large bore” (e.g., typically requiring a cannula with an inner diameter of at least 3 mm), which may be associated with higher rates of vascular complications than “small bore” sites (e.g., typically requiring a cannula with an inner diameter less than 3 mm).

Limb ischemia is a rapid and sudden decrease in limb perfusion often threatening limb viability. It may occur as a result of blockage of blood due to an indwelling sheath and/or catheter, local occlusion (e.g., atherosclerotic narrowing), and/or continuous occlusion resulting from small vessels and/or large sheaths. It may also occur as a result of a closure issue.

Limb ischemia may be associated with mortality, and some literature indicates the rate of limb ischemia may be high. As such, in addition to negatively impacting the patient, it may also negatively impact ongoing clinical trials.

### BRIEF SUMMARY

Various deficiencies in the prior art are addressed below by the disclosed systems.

In various embodiments, an unloading system may be provided. The unloading system may include a first cannula including a distal cannula body coupled to a proximal hub. The distal body may have a first inner diameter. The first cannula may be configured to slidably receive a mechanical circulatory support (MCS) device through the cannula. The first cannula may be configured to receive a return flow of blood through the first cannula. The system may include a second cannula. The second cannula may include a distal cannula body having an outer diameter smaller than the outer diameter of the first cannula. The second cannula may be configured to receive a return flow of blood through the second cannula. The system may include a connector for splitting a flow of blood from an extracorporeal membrane oxygenation (ECMO) device to the first cannula and the second cannula. The system may be configured such that a first total pressure drop of the first cannula with the MCS device in place in the first cannula with the second cannula connected to the connector may be equal to a second total pressure drop of the first cannula without the MCS device in place in the first cannula.

In various embodiments, the first inner diameter may be between 5 and 6 mm. In various embodiments, a thickness of a sidewall of the distal cannula body of the first cannula may be 0.1-0.3 mm. In various embodiments, the first cannula may include a thermoplastic polyurethane (TPU). In various embodiments, the first cannula may include one or more reinforcing coils coupled to the TPU. In various embodiments, the first cannula may include a laser-cut distal tip. In various embodiments, the first cannula may include one or more openings from an inner surface to an outer surface of the first cannula adjacent to a distal end.

In various embodiments, the proximal hub may include a first arm and a second arm. In various embodiments, the first arm may be configured to slidably receive the mechanical circulatory support (MCS) device. In various embodiments, the first arm may include a hemostasis valve. In various embodiments, the first arm may include a side-arm for flushing. In various embodiments, the second arm may include a barb connector for connection to an ECMO return. In various embodiments, the MCS device may have a maximum crossing profile of 4-6 mm during insertion. In various embodiments, the MCS device may have a catheter with an outer diameter of 2.5-3.5 mm.

In various embodiments, the second cannula may have an inner diameter equal to an outer diameter of a catheter of the MCS device. In various embodiments, the second cannula may include a thermoplastic polyurethane (TPU). In various embodiments, the second cannula may include one or more reinforcing coils coupled to the TPU. In various embodiments, the second cannula may include a laser-cut distal tip. In various embodiments, the second cannula may include one or more openings from an inner surface to an outer surface of the second cannula adjacent to a distal end.

In various embodiments, the system may include a third cannula operably coupled to the ECMO device and configured to pass blood from a patient to the ECMO device.

In various embodiments, a kit may be provided. The kit may include a first cannula including a distal cannula body coupled to a proximal hub, the distal body may have a first inner diameter, where the first cannula may be configured to slidably receive a mechanical circulatory support (MCS) device through the cannula and may be configured to receive a return flow of blood through the first cannula. The kit may include a second cannula including a distal cannula body having an outer diameter smaller than the outer diameter of the first cannula, the second cannula may be configured to receive a return flow of blood through the second cannula. The kit may include a connector for splitting a flow of blood from an extracorporeal membrane oxygenation (ECMO) device to the first cannula and the second cannula. The kit may include a first tubular member for coupling the connector to the proximal hub. The kit may include a second tubular member for coupling the connector to the second cannula. In various embodiments, the kit may include a third cannula configured to be inserted into a blood vessel to receive blood from a patient. In various embodiments, the kit may include an ECMO device.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is an illustration of an embodiment of a system.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention may also applicable to various other technical areas or embodiments.

To overcome the problems associated with multiple large bore sites, embodiments disclosed herein disclose concepts may to provide ECMO arterial return flow through the MCS sheath to combine access sites. In some embodiments, this may require a larger sheath diameter (associated with higher rates of vascular complications) for an equivalent pressure drop at a given flow rate compared to the current ECMO arterial return cannula or lower flow rates (that may not be sufficient therapy) for an equivalent pressure drop to the current ECMO arterial return cannula or a higher pressure drop (higher hemolysis generated by the centrifugal pump) for an equivalent flow to the current ECMO arterial return cannula.

A disclosed technique may avoid some or all of these problems. For example, in some embodiments, the disclosed technique may provide delivery of VA ECMO and MCS therapy, via a single ECMO venous cannula. In some embodiments, this technique may use the same diameter (large bore) sheath for MCS from a single access site for delivery of VA ECMO given the conditions of the alternative concept above. Further, an optional smaller diameter (small bore) cannula than current ECMO arterial return from a second access site may be used to deliver equivalent flow rates and overall cannula pressure drop as a current standard ECMO arterial return cannula. Such an approach ultimately provides equivalent VA ECMO and MCS therapy as can be provided today with a small bore ECMO arterial return cannula compared to current practice.

As will be appreciated, although describe for use with VA ECMO, the disclosed techniques may be used for other types of cannulation.

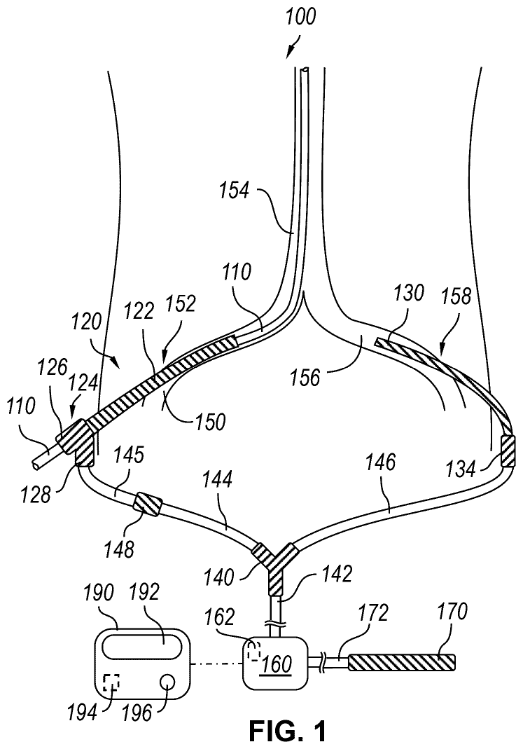

In some embodiment, an unloading system may be provided. Referring to FIG. 1, an unloading system 100 may include a first cannula 120. The first cannula may be configured to be inserted into a first blood vessel 150 (e.g., a right femoral artery) at a first insertion point 152.

The first cannula may include a distal cannula body 122 and a proximal hub 124. In some embodiments, the distal cannula body may include a tubular member with at least one sidewall defining at least one lumen extending from a proximal end to a distal end. The lumen may have a diameter (e.g., an inner diameter) that may be less than 6 mm. The inner diameter may be no more than 6 mm. The inner diameter may be no more than 5.9 mm. The inner diameter may be no more than 5.8 mm. The inner diameter may be no more than 5.7 mm. The inner diameter may be no more than 5.6 mm. The inner diameter may be no more than 5.5 mm. The inner diameter may be no more than 5 mm. The inner diameter may be 4-6 mm. The inner diameter may be 5-6 mm. The inner diameter may be 5.4-5.6 mm. In some embodiments, the distal cannula body may have an outer diameter that may be 6 mm or less.

In some embodiments, the sidewall may have a thickness of 0.1-0.5 mm, or between 0.1-0.3 mm. In some embodiments, the sidewall may have a thickness no more than 0.5 mm. In some embodiments, the sidewall may have a thickness no more than 0.45 mm. In some embodiments, the sidewall may have a thickness no more than 0.4 mm. In some embodiments, the sidewall may have a thickness no more than 0.35 mm. In some embodiments, the sidewall may have a thickness no more than 0.3 mm. In some embodiments, the sidewall may have a thickness no more than 0.25 mm. In some embodiments, the sidewall may have a thickness no more than 0.2 mm. In some embodiments, the sidewall may have a thickness no more than 0.15 mm. In some embodiments, the sidewall may have a thickness of at least 0.1 mm. In some embodiments, the sidewall may have a thickness of at least 0.15 mm. In some embodiments, the sidewall may have a thickness of at least 0.2 mm. In some embodiments, the sidewall may have a thickness of at least 0.25 mm. In some embodiments, the sidewall may have a thickness of at least 0.3 mm. In some embodiments, the sidewall may have a thickness of at least 0.35 mm. In some embodiments, the sidewall may have a thickness of at least 0.4 mm. In some embodiments, the sidewall may have a thickness of at least 0.45 mm.

The distal cannula body may include, e.g., a thermoplastic polyurethane (TPU). In some embodiments, the distal cannula body may include a reinforcing coil; for example, in some embodiments, the distal cannula body may include an inner wvall and an outer wall and a reinforced coil located between the inner wall and the outer wat.

In some embodiments, the first cannula may include a laser-cut distal tip. In some embodiments the cannula may include one or more openings extending from an inner surface to an outer surface (e.g., perpendicular to the central axis of the cannula body) of the distal cannula body, adjacent to the distal end of the cannula.

The proximal end of the distal cannula body may be connected to a distal end of the proximal hub. The proximal hub may be a proximal bifurcated hub. That is, extending proximally from the distal end, the hub may include a first arm or bifurcation 126 and a second arm or bifurcation 128.

The first arm or bifurcation may be configured to slidably receive a mechanical circulatory support (MCS) device 110. The first arm or bifurcation may include a hemostasis valve. The first arm or bifurcation may include a side-arm (not shown), e.g., for flushing.

The MCS device may be configured to be inserted through the first arterial cannula through the proximal hub and through the distal cannula body, before entering one or more blood vessels (e.g., first blood vessel 150, additional blood vessel 154). The MCS may have a crossing profile of less tar 5.5 am during insertion. In some embodiments, the MCS may have a crossing profile that may be 0.3-0.5 mm smaller than the inner diameter of the distal cannula body.

In some embodiments, a catheter of the MCS that remains within the distal cannula body during operation, may have an outer diameter of 4 mm or less. In some embodiments, the diameter may be 2.5-3.3 mm.

The second arm or bifurcation may include a connector (e.g., a barbed connector) configured to allow for connection to an ECMO return. That is, in some embodiments, the second arm or bifurcation may be configured to allow oxygenated blood to pass through the arm, into the distal cannula body, and then into the blood vessel.

The system may include a second cannula 130, The second cannula may be configured to be inserted into a second blood vessel 156 (e.g., a left femoral artery) at a second insertion point 158. The second cannula may include a distal cannula body. In some embodiments, the distal cannula body may include a tubular member with at least one sidewall defining at least one lumen extending from a proximal end to a distal end. The second cannula may have an outer diameter that may be smaller than the outer diameter of the first cannula.

The distal cannula body may have an inner diameter that may be approximately the same as the outer diameter of the catheter of the MCS. In some embodiments, the sidewall may have a thickness of 0.1-0.5 mm, or between 0.1-0.3 mm. In some embodiments, the sidewall may have a thickness of 0.25 mm. In some embodiments, the sidewall may have a thickness no more than 0.5 mm. In some embodiments, the sidewall may have a thickness no more than 0.45 mm. In some embodiments, the sidewall may have a thickness no more than 0.4 mm. In some embodiments, the sidewall may have a thickness no more than 0.35 mm. In some embodiments, the sidewall may have a thickness no more than 0.3 mm. In some embodiments, the sidewall may have a thickness no more than 0.25 mm. In some embodiments, the sidewall may have a thickness no more than 0.2 mm. In some embodiments, the sidewall may have a thickness no more than 0.15 mm. In some embodiments, the sidewall may have a thickness of at least 0.1 mm. In some embodiments, the sidewall may have a thickness of at least 0.15 mm. In some embodiments, the sidewall may have a thickness of at least 0.2 mm. In some embodiments, the sidewall may have a thickness of at least 0.25 mm. In some embodiments, the sidewall may have a thickness of at least 0.3 mm. In some embodiments, the sidewall may have a thickness of at least 0.35 mm. In some embodiments, the sidewall may have a thickness of at least 0.4 mm. In some embodiments, the sidewall may have a thickness of at least 0.45 mm.

The distal cannula body may include, e.g., a thermoplastic polyurethane (TPU). In some embodiments, the distal cannula body may include a reinforcing coil; for example, in some embodiments, the distal cannula body may include an inner wall and an outer wall and a reinforced coil located between the inner wall and the outer wall. The reinforcing coil may be a metal coil. The reinforcing coil may have a constant pitch length. The reinforcing coil may have a variable pitch length.

In some embodiments, the second cannula may include a shaped distal tip. In some embodiments, the second cannula may include a laser-cut distal tip. In some embodiments, the cannula may include one or more openings extending from an inner surface to an outer surface (e.g., perpendicular to the central axis of the cannula body) of the distal cannula body, adjacent to the distal end of the cannula.

The second cannula may be coupled to a connector 134 configured for receiving an ECMO return. That is, in some embodiments, the second cannula may be configured to allow oxygenated blood to pass through the distal cannula body, and then into the blood vessel.

The system may include a connector 140. In some embodiments, the connector may be a three-way connector. In some embodiments, the connector may be a Y-connector. In some embodiments, the connector may be a T-connector. In some embodiments, the connector may be a V-connector. In some embodiments, the connector may be configured to be connected to an inlet tube 142 for receiving an ECMO return flow. In some embodiments, the connector may be configured to be connected to an outlet tube 144 for transferring the ECMO return flow from the connector 140 to the proximal hub (e.g., the second arm 128 of the proximal hub 124) of the first cannula. In some embodiments, the connector may be configured to be connected to an outlet tube 146 for transferring the ECMO return flow from the connector 140 to the second cannula 130.

The system may include a one-way value 148. The one-way value may be disposed between two tubes (in FIG. 1, this is shown as being disposed between, e.g., outlet tube 144 and additional tube 145).

In various embodiments, the system may be configured such that the total pressure drop of the first cannula 120 with an indwelling MCS device 110 when the second cannula 130 is connected to the connector 140 being equal to the first cannula 120 without the indwelling MCS.

In some embodiments, the inlet tube 142 may be operably coupled to at least one external device 160. In some embodiments, the external device may include, e.g., an ECMO device, a heat exchanger, a pump, etc. In some embodiments, the external device may include a sensor 162. The sensor may be, e.g., a pressure sensor, a flow sensor, a temperature sensor, a voltage sensor, or a combination thereof.

The external device may be operably coupled to a controller 190. The controller may include a display screen 192. The controller may include one or more processing units 194. The controller may include one or more buttons 196. The controller may be configured to control the external device. The controller may be configured to receive information from one or more sensors, such as a sensor 162 in an external device.

In some embodiments, the external device may be coupled to a controller (not shown).

The unloading system may also include a third cannula 170. The second cannula may be configured to be inserted into a second blood vessel (e.g., a vein) at a third insertion point (not shown). The third cannula may be configured to receive blood from a patient, before passing it to, e.g., an ECMO device and then back to the first and/or second cannulas. The third cannula may be operably coupled to an inlet tube 172. The inlet tube may be operably coupled to an external device.

The third cannula may include a distal cannula body. In some embodiments, the distal cannula body may include a tubular member with at least one sidewall defining at least one lumen extending from a proximal end to a distal end.

In some embodiments, the sidewall may have a thickness of 0.1-0.5 mm, or between 0.1-0.3 mm. In some embodiments, the sidewall may have a thickness no more than 0.5 mm. In some embodiments, the sidewall may have a thickness no more than 0.45 mm. In some embodiments, the sidewall may have a thickness no more than 0.4 mm. In some embodiments, the sidewall may have a thickness no more than 0.35 mm. In some embodiments, the sidewall may have a thickness no more than 0.3 mm. In some embodiments, the sidewall may have a thickness no more than 0.25 mm. In some embodiments, the sidewall may have a thickness no more than 0.2 mm. In some embodiments, the sidewall may have a thickness no more than 0.15 mm. In some embodiments, the sidewall may have a thickness of at least 0.1 mm. In some embodiments, the sidewall may have a thickness of at least 0.15 mm. In some embodiments, the sidewall may have a thickness of at least 0.2 mm. In some embodiments, the sidewall may have a thickness of at least 0.25 mm. In some embodiments, the sidewall may have a thickness of at least 0.3 mm. In some embodiments, the sidewall may have a thickness of at least 0.35 mm. In some embodiments, the sidewall may have a thickness of at least 0.4 mm. In some embodiments, the sidewall may have a thickness of at least 0.45 mm.

The distal cannula body may include, e.g., a thermoplastic polyurethane (TPU). In some embodiments, the distal cannula body may include a reinforcing coil; for example, in some embodiments, the distal cannula body may include an inner wall and an outer wall and a reinforced coil located between the inner wall and the outer wall.

In some embodiments, the third cannula may include a laser-cut distal tip. In some embodiments, the cannula may include one or more openings extending from an inner surface to an outer surface (e.g., perpendicular to the central axis of the cannula body) of the distal cannula body, adjacent to the distal end of the cannula.

Various modifications may be made to the systems, methods, apparatus, mechanisms, techniques and portions thereof described herein with respect to the various figures, such modifications being contemplated as being within the scope of the invention. For example, while a specific order of steps or arrangement of functional elements may be presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing may be directed to various embodiments of the present invention, other and further embodiments of the invention may be devised without departing from the basic scope thereof. As such, the appropriate scope of the invention is to be determined of the claims.

1. An unloading system comprising:
a first cannula including a distal cannula body coupled to a proximal hub, the distal cannula body having a first inner diameter, where the first cannula is configured to slidably receive a mechanical circulatory support (MCS) device through the first cannula, and configured to receive a return flow of blood through the first cannula;
a second cannula including a distal cannula body having an outer diameter smaller than the outer diameter of the first cannula, the second cannula configured to receive a return flow of blood through the second cannula; and
a connector for splitting a flow of blood from an extracorporeal membrane oxygenation (ECMO) device to the first cannula and the second cannula;
wherein a first total pressure drop of the first cannula with the MCS device in place in the first cannula with the second cannula connected to the connector is equal to a second total pressure drop of the first cannula without the MCS device in place in the first cannula.

2. The unloading system of claim 1, wherein the first inner diameter is between 5 and 6 mm.

3. The unloading system of claim 1, wherein a thickness of a sidewall of the distal cannula body is 0.1-0.3 mm.

4. The unloading system of claim 1, wherein the first cannula includes a thermoplastic polyurethane (TPU).

5. The unloading system of claim 4, wherein the first cannula further comprises one or more reinforcing coils coupled to the TPU.

6. The unloading system of claim 1, wherein the first cannula comprises a laser-cut distal tip.

7. The unloading system of claim 1, wherein the first cannula comprises one or more openings from an inner surface to an outer surface of the first cannula adjacent to a distal end.

8. The unloading system of claim 1, wherein the proximal hub comprises a first arm and a second arm.

9. The unloading system of claim 8, wherein the first arm is configured to slidably receive the mechanical circulatory support (MCS) device.

10. The unloading system of claim 8, wherein the first arm comprises a hemostasis valve.

11. The unloading system of claim 8, wherein the first arm comprises a side-arm for flushing.

12. The unloading system of claim 8, wherein the second arm includes a barb connector for connection to an ECMO return.

13. The unloading system of claim 1, wherein the MCS device has a maximum crossing profile of 4-6 mm during insertion.

14. The unloading system of claim 1, wherein the MCS device has a catheter with an outer diameter of 2.5-3.5 mm.

15. The unloading system of claim 1, wherein the second cannula has an inner diameter equal to an outer diameter of a catheter of the MCS device.

16. The unloading system of claim 1, wherein the second cannula includes a thermoplastic polyurethane (TPU).

17. The unloading system of claim 16, wherein the second cannula further comprises one or more reinforcing coils coupled to the TPU.

18. The unloading system of claim 16, wherein the second cannula comprises a laser-cut distal tip.

19. The unloading system of claim 1, wherein the second cannula comprises one or more openings from an inner surface to an outer surface of the second cannula adjacent to a distal end.

20. The unloading system of claim 1, further comprising a third cannula operably coupled to the ECMO device and configured to pass blood from a patient to the ECMO device.

21. A kit, comprising:
a first cannula including a distal cannula body coupled to a proximal hub, the distal cannula body having a first inner diameter, where the first cannula is configured to slidably receive a mechanical circulatory support (MCS) device through the first cannula, and configured to receive a return flow of blood through the first cannula;
a second cannula including a distal cannula body having an outer diameter smaller than the outer diameter of the first cannula, the second cannula configured to receive a return flow of blood through the second cannula;
a connector for splitting a flow of blood from an extracorporeal membrane oxygenation (ECMO) device to the first cannula and the second cannula;
a first tubular member for coupling the connector to the proximal hub; and
a second tubular member for coupling the connector to the second cannula.

22. The kit of claim 21, further comprising a third cannula configured to be inserted into a blood vessel to receive blood from a patient.

23. The kit of claim 21, further comprising an ECMO device.
